(19) United States
(12) Patent Application Publication
(10) Pub. No.: US 2004/0035415 A1
(43) Pub. Date: Feb. 26, 2004

(54) Breathing apparatus

(76) Inventor: Michel Faligant, The Valley (AI)

Correspondence Address: DIEDERIKS & WHITELAW, PLC, 12471 Dillingham Square, #301, Woodbridge, VA, 22192, US

(21) Appl. No.: 10/225,236

(22) Filed: Aug. 22, 2002

(51) Int. Cl. A61M 16/00; B63C 11/02

(52) U.S. Cl. 128/201.27; 128/201.28; 128/204.26

(57) ABSTRACT

A breathing apparatus for a diver is configured to include a second stage pressure regulator and a hollow exhaust tube. An input port is located on a side of the pressure regulator and is attached to a supply hose coming from a tank mounted on a scuba diver's back. Additionally, an exhaust port is located on the same side of the regulator as the input port. The hollow exhaust tube is attached to the exhaust port. The exhaust tube is able to trap gas therein by the placement of one-way valves at each end of the exhaust tube, thus supplying a counteracting buoyant force to the same side of the regulator as the side where the supply hose enters the regulator, while defining an exhaust port arranged so that bubbles leaving the exhaust tube do not obstruct the diver's vision. The regulator may be fit with a supporting surface designed to rest on a diver's chin.

### BACKGROUND OF THE INVENTION

1. Field of the Invention

The present invention generally pertains generally to the art of self-contained underwater breathing apparatus (SCUBA). More specifically, the invention concerns a device for reducing the amount of force which must be supplied at a diver's mouth while a breathing apparatus is in use and, additionally, structure for diverting exhaust bubbles away from the diver's face while reducing produced vibrations.

2. Discussion of the Prior Art

A typical self-contained underwater breathing apparatus or scuba arrangement includes many functional parts which are designed to work together to provide air or other breathable gas to a diver while he is underwater. First air or other suitable gas is typically stored in a high-pressure tank supported by a harness designed to mount on a scuba diver's back. Typically the tank is made either of aluminum or steel and can handle gas pressures of approximately 3000 psi. The tank or bottle has a single port in which is mounted a high-pressure tank valve. A first stage pressure regulator is mounted to the high pressure tank valve and functions to reduce the supplied gas pressure to about 150 psi. A flexible supply hose is connected at one end to the first stage regulator and carries the reduced pressure gas to a second stage regulator typically mounted in front of the diver's mouth and supported by a mouthpiece. The mouthpiece has some type of tabs to allow for the diver to bite down upon the mouthpiece and thereby support the second stage regulator in the diver's mouth. The weight of the supply hose and force supplied through the hose from the tank all work to dislodge the regulator from the diver's mouth. A diver must spend considerable effort and use a strong bite to maintain the second stage regulator in the proper position. This of course is very uncomfortable and fatiguing to the diver.

The second stage regulator typically will provide pressurized gas when the diver inhales. This function is accomplished by means of a diaphragm switch which activates a lever to open a valve controlling the flow of gas to the diver. When the diver stops inhaling, or alternatively is exhaling, the diaphragm senses an increase in gas pressure and moves accordingly to close the valve and stop the flow of gas. A separate exhaust valve or port is provided to allow for the escape of exhausted gases. The valve prevents water from entering the second stage regulator when the diver is inhaling. The exhaust gases tend to leave the diver's regulator as bubbles, most often quite close to the diver's face thus obstructing the diver's view. This can be particularly bothersome in underwater photography wherein photographers will frequently have to time their breathing in order to avoid having a photograph filled with bubbles or distorted by vibration.

In an attempt to overcome these deficiencies, several solutions have been proposed. For example, U.S. Pat. No. 3,721,235 discloses using an extendible flexible extension 15b which transfers exhaust bubbles from a mouthpiece exhaust port 10a to a relatively remote venting port 15d located away from the diver's face. However, while this arrangement does remove the bubbles from the diver's face, it does nothing to address the problem of the large forces which act upon the second stage regulator and, more particularly, the diver's mouthpiece. Indeed with such a relatively long flexible tube as proposed in U.S. Pat. No. 3,721,235 being carried in the water behind the diver, the force on the mouthpiece could increase dramatically due to the resistance of the water acting on the tube while the diver is swimming.

U.S. Pat. No. 4,467,797 discloses an embodiment of a regulator wherein the amount of exhalation and inhalation effort produced by the diver is reduced. Additionally there is provided an exhaust conduit 40 that permits exhaust venting somewhat remote from the diver's face. However, no where in this patent is there disclosed either the problem or a solution to counter the various forces that would be placed on the mouthpiece given this arrangement.

Therefore, there exists a need in the art for an arrangement of a second stage pressure regulator which will direct exhaust bubbles away from a diver's face and simultaneously increase a diver's comfort level by reducing the amount of force on the second stage regulator's mouthpiece. Additionally, there exists a need in the art for such a device to be easily attached to or integrated with currently available scuba regulators so that a diver may obtain these benefits without having to purchase an entirely new regulator.

### SUMMARY OF THE INVENTION

In accordance with the present invention, a breathing apparatus is configured to include a second stage regulator and a hollow exhaust tube. The second stage regulator has a housing from which a mouthpiece extends to allow gas or air to be supplied to a diver. An input port is located on the side of the second stage regulator and is attached to a gas supply hose coming from a first stage regulator secured to a tank mounted on the scuba diver's back. The supply hose is relatively heavy, receives gas from the gas source or tank located on the scuba diver's back, and directs the gas to the second stage regulator housing. Additionally, an exhaust port is located on the same side of the regulator as the input port. A hollow exhaust tube, which may be of variable length and cross-section, is attached to the exhaust port. The exhaust tube is designed to trap gas therein, thus supplying a controlled amount of buoyant force to the same side of the regulator as the side where the supply hose enters the regulator.

With this arrangement, the buoyancy force from the gas in the exhaust tube counteracts the weight of the supply hose so less twisting force is supplied through the mouthpiece to the diver. Preferably the exhaust tube has a first end attached to the exhaust port, a long hollow central portion, and a second end located remote from the exhaust port so that bubbles leaving the exhaust tube do not obstruct the diver's vision. The exhaust tube is attached to the supply hose by one or more hooks that are preferably integrally formed with the exhaust tube. Alternatively, the exhaust tube may be attached to the supply hose by a wire tie.

In a second embodiment, the breathing apparatus is arranged to be a retrofit unit. In this arrangement, a standard second stage regulator having two housings that are detachable from one another is modified to incorporate the present invention. The first part of the housing in a typical regulator arrangement includes a mouthpiece along with various operating parts such as a diaphragm and lever actuated valve, and also includes an input port to be connected to the supply hose for receiving gas. The exhaust housing is detachably mounted to the main housing and includes ports for exhausting exhaled air. The invention provides for a retrofit exhaust housing having an exhaust tube mounted thereto in a similar manner to the first embodiment. Essentially, once the retrofit housing is attached to the original main housing, a unit is formed similar to the first embodiment. A gasket seals the two housings. The exhaust tube is attached to the supply hose by a hook integrally formed with the exhaust tube. Alternatively, the exhaust tube may be attached to the supply hose by a wire tie. In order to improve gas flow through the exhaust tube, includes a hollow central portion made of a flexible plastic material having a smooth interior. Preferably the exterior of the tube is corrugated to provide additional strength. The exhaust tube preferably has one-way valves, one located at each end in order to aid in trapping gas within the hollow tube, however, the valve closest to the second stage regulator is optional.

In a variation of both the first and second embodiments, the regulator is fit with a resting or support surface designed to rest on a diver's chin, thereby stabilizing the second stage regulator by obtaining additional support for the housing from the diver's chin. This support surface can either be directly mounted on the second stage regulator as in the first embodiment above or, alternatively, may be mounted on the retrofit unit. If the surface is mounted on the retrofit housing, then a standard second stage regulator may be modified by the invention to obtain the invention's advantages without having a diver purchase an entire new unit. The support surface may be a modular piece, one of many such pieces of differing shapes preferably adhered to the second stage regulator or retrofit unit such as by an adhesive.

Additional objects, features and advantages of the present invention will become more readily apparent from the following detailed description of preferred embodiments when taken in conjunction with the drawings wherein like reference numerals refer to corresponding parts in the several views.

### BRIEF DESCRIPTION OF THE DRAWINGS

FIG. 1 is a side elevation view showing a typical diver in an underwater environment using scuba gear equipped with the present invention;

FIG. 2 is a close-up view of a diver using a second stage regulator equipped with a preferred embodiment of the invention;

FIG. 3 is a perspective view of a second stage regulator equipped with a second preferred embodiment of the invention;

FIG. 4 is a perspective view of the regulator shown in FIG. 2;

FIG. 5 is an expanded view of a regulator according to another preferred embodiment of the invention;

FIG. 6 is a perspective view of an exhaust tube according to another preferred embodiment of the invention;

FIG. 7 is a perspective view of the exhaust tube of FIG. 6 attached to a second stage regulator;

FIG. 8 is a perspective view of a regulator found in the prior art;

FIG. 9 is a side view of a regulator incorporating a chin support surface according to yet another embodiment of the invention; and

FIG. 10 is a side view of a regulator having a retro-fit housing and incorporating a chin support surface according to yet another preferred embodiment of the invention.

### DETAILED DESCRIPTION OF THE PREFERRED EMBODIMENTS

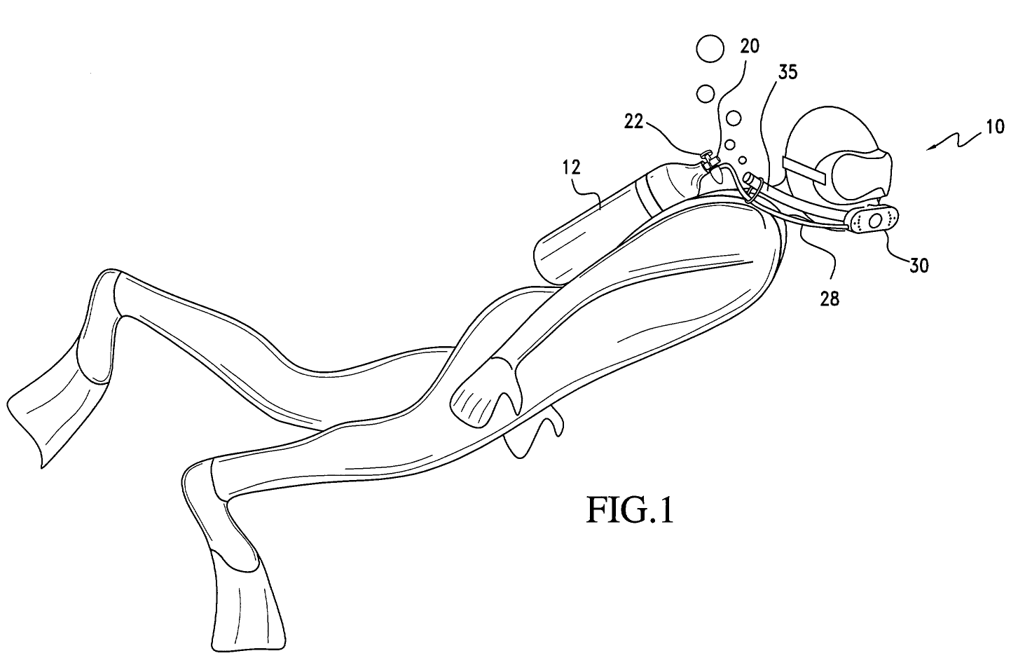

With initial reference to FIG. 1, there is shown a diver 10 swimming underwater equipped with a self-contained underwater breathing apparatus according to a first embodiment of the invention. The breathing apparatus or scuba gear includes a tank 12 that stores air or other breathable gas under extremely high pressure. Tank 12 is supported by a harness (not shown) and mounted on the scuba diver's back. Typically, tank 12, which includes a tank valve 20, is made of either aluminum or steel and is designed to contain gas at pressures of approximately 3000 PSI, with the gas being slowly released to provide air to diver 10 during a dive. A first stage pressure regulator 22 is mounted to tank 12 via the high-pressure tank valve 20. First stage pressure regulator 22 functions to reduce the supply of gas pressure to about 150 psi. First stage pressure regulator 22 is easily removed from tank valve 20 to allow tank 12 to be filled and is securely attached to tank 12 during a dive. A flexible supply hose 28 is connected at one end to first stage regulator 22 and carries reduced pressure gas to a second stage regulator 30 mounted in front of the diver's mouth. Also mounted to second stage regulator 30 is an exhaust tube 35, which is designed to direct bubbles away from the diver's face and attached to supply hose 28.

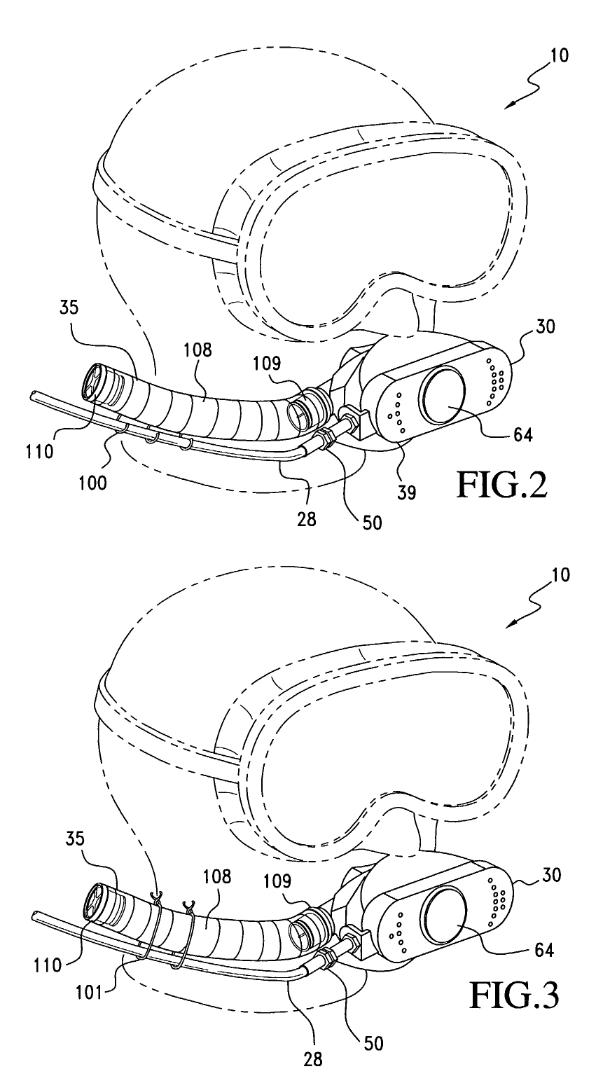

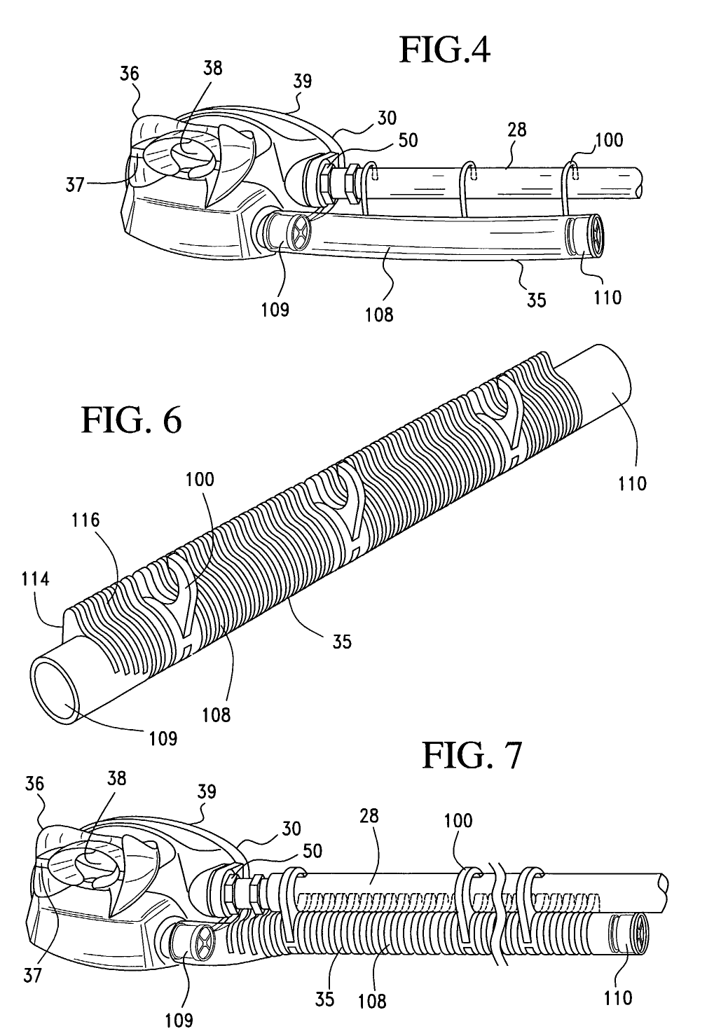

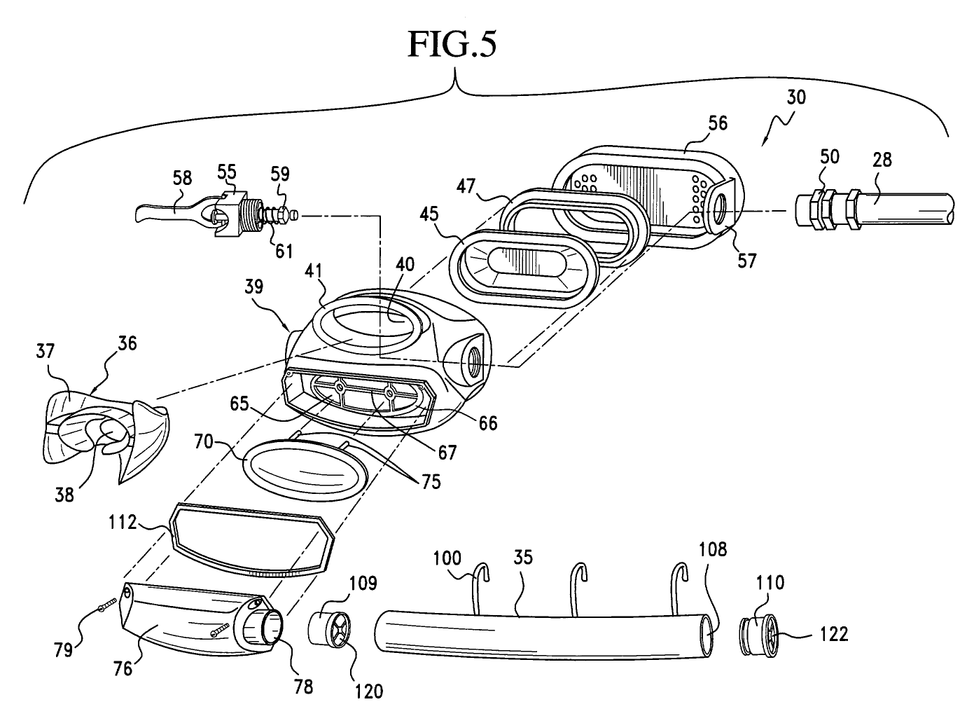

Turning now to FIGS. 2-6, there is shown the second stage regulator 30 of FIG. 1 in further detail. Flexible supply hose 28 is relatively heavy and tends to pull on second stage pressure regulator 30. A mouthpiece 36 is provided which both allows diver 10 to hold the second stage pressure regulator 30 in place and provides for air from tank 12. In order to hold second stage pressure regulator 30 in place, mouthpiece 36 is provided with clamping tabs 37 (see FIG. 4) designed to be easily held by the diver's teeth. Mouthpiece 36 also has an internal conduit 38 to allow for diver 10 to breathe while biting down on mouthpiece 36. Conduit 38 leads into main housing 39 of second stage regulator 30. As seen in FIG. 5, main housing 39 includes a breathing port 40 having an oval cross section and a mounting flange 41 designed to allow mouthpiece 36 to be removably attached thereto. Opposite the breathing port 40 is a diaphragm 45 that is secured across a majority of main housing 39. A securing ring 47 is provided around main housing 39 to seal the edges of diaphragm 45 to main housing 39.

As stated above, supply hose 28 provides relatively high pressure air to the second stage regulator 30. Supply hose 28 is first attached to a swivel connection 50 to enhance the positioning of second pressure regulator 30 in the diver's mouth. Swivel connection 50 is threaded to a valve 55 located inside main housing 39. A cover 56 is mounted over diaphragm 45. Cover 56 has a securing tab 57 that wraps around main housing 39 and is held in place by being trapped between swivel connection 50 and main housing 39. Valve 55 is physically located in main housing 39 and is actuated by a lever 58 contacting diaphragm 45. Lever 58 is preferably constituted by a thin metal elongated flexible member which is pivotably attached to valve 55. Valve 55 includes a stem 59 with a poppet seat insert 60 which is biased by a spring 61. A lever support 62 pivotably supports lever 58. Additionally there is a purge button 64 or actuation switch that can manually activate lever 58 and thus valve 55 of second stage regulator 30. Button 64 may either be a flexible portion of cover 56 or a separately movable actuation member.

When second stage pressure regulator 30 is dropped by diver 10 and second stage pressure regulator 30 floods with water, diver 10 has two basic options. Diver 10 may replace second stage pressure regulator 30 in the diver's mouth and exhale thus clearing the second stage regulator 30 as water is forced out exhaust tube 35. Otherwise diver 10 may press purge button 64 to allow high pressure air tend to second stage regulator 30 from the supply and thus venting second stage regulator 30. A one way exhaust valve 65 is provided in main housing 39. One way exhaust valve 65 is mounted over an opening 66 having an open lattice grid 67. A rubber oval flexible member 70 is mounted over grid 67 and secured in place by pins 75. As air pushes in one direction flexible member 70 is pulled away from grid 67 and allows for the passage of air. When the air flows in the other direction flexible member 70 is pushed against grid 67 and thus stops the airflow.

An exhaust housing 76 is mounted over one way valve 65 and exhaust housing 76 is provided with an exhaust port 78. Exhaust housing 76 may be integrally formed with main housing 39 or detachably mounted thereto by screws 79. Exhaust tube 35 is attached to supply hose 28 by one or more hooks 100 that are preferably integrally formed with exhaust tube 35. Alternatively, as shown in FIG. 3, exhaust tube 35 may be attached to supply hose 28 by one ore more wire ties 101. In order to improve gas flow through exhaust tube 35, tube 35 is provided with a hollow central portion 108 is preferably made of a flexible plastic material having a smooth interior. Preferably the exterior of tube 35 is corrugated to provide additional strength. The exhaust tube 35 may be designed with different lengths or of different cross section thus changing the amount of gas held therein.

Since the buoyant force provided by tube 35 is controlled by the tube's volume, tube 35 should be sized to compensate for the weight of supply hose 28. Ideally the weight of supply hose 28 will be canceled by the buoyancy of tube 35. Exhaust tube 35 preferably has two one way valves 109, 110, one located at each end in order to aid in trapping gas within hollow central portion 108. However, valve 109 is optional. Exhaust tube 35 is placed in fluid communication with exhaust port 78 on exhaust housing 76 and extends away from exhaust housing 76 along the same path as supply hose 28. As mentioned above, main housing 39 and exhaust housing 76 may be integrally formed as shown in FIG. 4 or may be two separate detachable pieces as shown in FIG. 5. In the case of a detachably mounted arrangement, a seal or gasket 112 is formed between main housing 39 and exhaust housing 76.

Turning now to FIG. 6, there can be seen an exhaust tube 35 having two one-way valves 109 and 110, one located at each end of the hollow central portion 108 in a manner similar to that shown in FIG. 4. However, in the embodiment shown in FIG. 6, there is additionally provided an abutment 114 formed as part of tube 35 which extends along the entire length of tube 35. Abutment 114 has a concave surface 116. As seen in FIG. 7, concave surface 116 and hooks 100 cooperate to trap supply hose 28 snugly against tube 35. With this configuration, when tube 35 is attached to hose 28, tube 35 and hose 28 are held tightly together in a snug manner and thus reduce the amount of resistance a diver feels when swimming.

In normal use, diver 10 holds mouthpiece 36 in his mouth so that when he inhales the pressure on one side of diaphragm 45 inside main housing 39 is lowered. This lower pressure actuates lever 58 that opens valve 55 regulating the air supplied to second stage pressure regulator 30 from supply hose 28. Air then travels through second stage pressure regulator 30 and mouthpiece 36 into the diver's lungs. When diver 10 exhales, diaphragm 45 is moved in the opposite direction thus shutting off valve 55 at the inlet to second pressure stage regulator 30. Further exhalation forces air out of exhaust port 78 on second stage regulator 30 and passes first optional one-way valve 109 and into elongated hollow central portion 108. Air finally exits out of second one-way valve 110. Typically these one-way valves 109, 110 are formed with a flexible flap 120,122 made of rubber or other suitable material. Flaps 120,122 are held in normally a closed position. Pressure from one end of one of flaps 120,122 simply allows flaps 120,122 to open and allow air pressure to travel through a respective one way valve 109, 110. However, when the flow of fluid is in the other direction, the one way valves 109, 110 will simply close thereby prohibiting any further flow of air.

During normal operation, exhaust tube 35 will become full of air or other breathable gas. As such, it will have a buoyant force. Supply hose 28 going from scuba tank 12 to first stage regulator to second stage regulator 30 will naturally have a certain weight and thus also apply a force to second stage regulator 30. As can best be seen in FIG. 4, hooks 100 are integrally molded into exhaust tube 35 and are also attached to supply hose 28. The interaction of these hooks 100, the buoyant force of exhaust tube 35 and the weight of supply hose 28 all work to counteract one another and thus avoid twisting action on second stage pressure regulator 30. As such, there is much less of a force applied to diver 10 through mouthpiece 36 as diver 10 tries to hold second stage pressure regulator 30 in place while breathing.

As mentioned above, the amount of buoyant force provided can be adjusted by changing the size and shape of tube 35. As mentioned above, in an alternative embodiment, as shown in FIG. 3, exhaust tube 35 may be held together with supply hose 28 by means of wire tie 101 which serve the same function as hook 100. The details of the airflow throughout this process can probably best be seen in FIG. 4. In addition to the force of supply hose 28 on the diver's mouth being lessened, there is also an added benefit that bubbles leaving from exhaust tube 35 will be exhausted at a point remote from the diver's face. Unlike in the prior art device where these bubbles move in front of the diver's face and thus cause distraction, especially in underwater photography. With the addition of exhaust tube 35 no such problems occur.

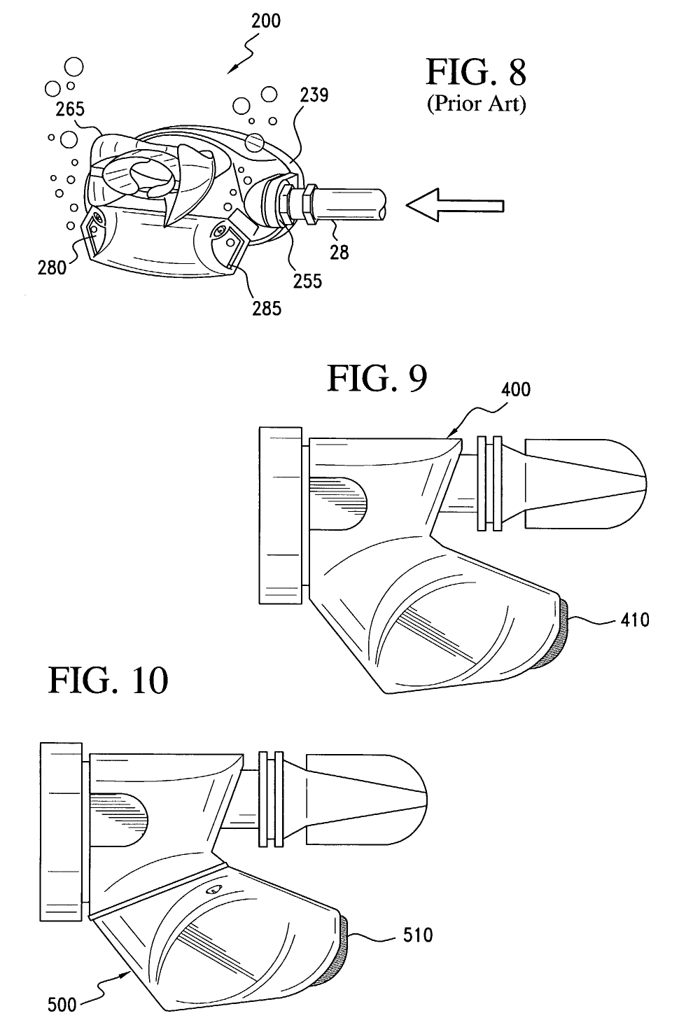

The problem of bubbles leaving a typical prior art second stage regulator 200 can be seen by the air flow diagram shown in FIG. 8. Regulator 200 works in a similar manner to the regulator discussed in regards to the embodiment shown in FIG. 4 and has an input valve that is actuated by a lever moved by a diaphragm (all not shown). Such a regulator 200 is currently made by Sherwood Scuba and sold as model Brut SRB5100. Essentially, a two part housing is provided. A first housing 239 includes the valving arrangement such as a diaphragm and a lever actuated valve and an input port 255 connected to supply hose 28. Additionally, an opening for a mouthpiece 265 is provided in order to supply gas to diver 10. A second housing 276, an exhaust housing 276, is detachably mounted to the main housing 239. Exhaust housing 276 has a pair of output ports 280, 285 that allow for escaping gas.

Turning now to FIG. 9, there is shown a second stage regulator 400 which in most respects is identical to that shown in FIG. 4 with the exception that it has an additional custom fit chin supporting surface 410 glued or otherwise secured below mouthpiece 39. Chin supporting surface 410 is designed to abut against a diver's chin during use. As such chin supporting surface 410 provides a force provided from the diver's chin to stabilize second stage regulator 400 reducing vibration and other sorts of discomfort. Ideally, several custom fit supporting surface 410 pieces would be available at a store and customers could try them to see which provides a better fit. The chosen surface would then be glued to the regulator 400.

In FIG. 10, a similar configuration is shown wherein a chin supporting surface 510 is mounted on a retrofit housing 500. This arrangement is similar to the two part regulator shown in FIG. 5 except for of course the addition of chin supporting surface 510. Once again, chin support surface 510 will rest against the diver's chin during use and counteract any turbulence or other undesirable motion of the regulator 500. It is important to note that since chin support surface 510 is on the retrofit unit, such housing may be added to any existing regulator device 200.

Although described with reference to preferred embodiments of the invention, it should be readily understood that various changes and/or modifications could be made to the invention without departing from the spirit thereof. In general, the invention is only intended to be limited by the scope of the following claims.

I/we claim:

1. An underwater breathing apparatus comprising:
a pressure regulator including a housing;
an input port located on a first side of the pressure regulator being adapted to be attached to a supply hose receiving gas from a gas source;
a mouthpiece extending from said housing and allowing for the gas to be supplied to a diver;
an exhaust port located on the first side of the pressure regulator;
a hollow exhaust tube attached to the exhaust port, said exhaust tube being adapted to trap gas therein, thereby supplying a buoyant force to the first side of the pressure regulator; and
whereby, when a supply hose is attached to the input port of the pressure regulator, the buoyant force created in the exhaust tube counteracts a tendency of the mouthpiece to be twisted by the supply hose.

2. The breathing apparatus of claim 1, further comprising a supply hose attached to said input port and wherein the exhaust tube has a first end portion attached to the exhaust port, a long hollow central portion, and a second end portion located remote from the exhaust port, said exhaust tube being further attached to said supply hose.

3. The breathing apparatus according to claim 2, further comprising a hook mounted on said exhaust tube and attached to said supply hose.

4. The breathing apparatus according to claim 2, further comprising a plurality of hooks all integrally formed with said exhaust tube and attached to said supply hose.

5. The breathing apparatus according to claim 2, further comprising a wire tie attached to said exhaust tube and attached to said supply hose.

6. The breathing apparatus according to claim 2, wherein said long hollow central portion of the exhaust tube is made of a flexible material having a smooth interior to enhance gas flow and a corrugated exterior to enhance tube strength.

7. The breathing apparatus according to claim 2, further comprising a first one-way valve located at said first end portion and a second one-way valve located at said second end portion, said first and second one-way valves working to trap gas in the hollow exhaust tube, thus providing the buoyant force.

8. The breathing apparatus according to claim 1, wherein the exhaust tube has a length and a cross-section which establishes a predetermined volume, said predetermined volume enabling the buoyant force to effectively counter the tendency of the mouthpiece to be twisted by the supply hose.

9. An underwater breathing apparatus comprising:
a pressure regulator including a main housing and an exhaust housing;
an input port located on the main housing on a first side of the pressure regulator, said input port being adapted to be attached to a supply hose receiving gas from a gas source;
a mouthpiece extending from said main housing and allowing for the gas to be supplied to a diver;
an exhaust port located on the exhaust housing on the first side of the pressure regulator; and
a hollow exhaust tube having a first end portion attached to the exhaust port, a long hollow central portion, and a second end portion located remote from the exhaust port, said exhaust tube being adapted to trap gas therein to create a buoyant force and being adapted to be attached to a supply hose;
whereby, when a supply hose is attached to the input port of the pressure regulator, the buoyant force in the exhaust tube counteracts a tendency of the mouthpiece to be twisted by the supply hose.

10. The breathing apparatus according to claim 9, further comprising a hook mounted on said exhaust tube and adapted to be attached to a supply hose.

11. The breathing apparatus according to claim 9, further comprising a plurality of hooks integrally formed with said exhaust tube and an abutment integrally formed with said exhaust tube, said plurality of hooks and said abutment adapted to work together to attach said exhaust tube to a supply hose in a snug manner.

12. The breathing apparatus according to claim 9, wherein said long hollow central portion of the exhaust tube is made of a flexible material having a smooth interior to enhance gas flow and a corrugated exterior to enhance tube strength.

13. The breathing apparatus according to claim 9, further comprising a first one-way valve located at said first end portion and a second one-way valve located at said second end portion, said first and second one-way valves working to trap gas in the hollow exhaust tube, thus providing the buoyant force.

14. The breathing apparatus according to claim 9, wherein the exhaust tube has a length and a cross-section which establishes a predetermined volume, said predetermined volume enabling the buoyant force to effectively counter the tendency of the mouthpiece to be twisted by the supply hose.

15. The breathing apparatus according to claim 9, wherein said main housing and said exhaust housing are integrally formed.

16. The breathing apparatus according to claim 9, further comprising:
a gasket located between the main housing and the exhaust housing; and
a pair of screws interconnecting the exhaust housing and the main housing.

17. An exhaust assembly for an underwater breathing apparatus comprising:
an exhaust housing;
an exhaust port located on the exhaust housing; and
a hollow exhaust tube having a first end portion attached to the exhaust port, a long hollow central portion and a second end portion located remote from the exhaust port, said exhaust tube being adapted to trap gas therein to create a buoyant force and being adapted to be attached to a supply hose.

18. The exhaust assembly according to claim 17, further comprising a plurality of hooks integrally formed with said exhaust tube and an abutment integrally formed with said exhaust tube, said plurality of hooks and said abutment adapted to work together to attach said exhaust tube to a supply hose in a snug manner.

19. A breathing apparatus for use underwater comprising;
a pressure regulator including a housing;
a mouthpiece extending from said housing and allowing for gas to be supplied to a diver;
an input port adapted to be attached to a supply hose to receive gas from a gas source and allowing the gas to enter the housing;
an exhaust port for allowing gas to leave the housing; and
a supporting surface extending below said mouthpiece and being adapted to abut a diver's chin, wherein force from the diver's chin helps to support the housing and reduce the amount of force applied to the diver from the mouthpiece.

20. The breathing apparatus according to claim 19, wherein said supporting surface is attached to said pressure regulator by an adhesive.
